(19) United States
(12) Patent Application Publication
(10) Pub. No.: US 2026/0108165 A1
(43) Pub. Date: Apr. 23, 2026

(54) SYSTEM AND METHOD FOR CELLULAR DECONVOLUTION USING MAGNETIC RESONANCE IMAGING (MRI)

(71) Applicant: Wisconsin Alumni Research Foundation, Madison, WI (US)

(72) Inventors: John-Paul Jaewoon Yu, Madison, WI (US); Luis Arnoldo Vazquez, El Paso, TX (US)

(21) Appl. No.: 18/920,850

(22) Filed: Oct. 18, 2024

(51) Int. Cl. A61B 5/055; A61B 5/00; G01R 33/56

(52) U.S. Cl. CPC A61B 5/055; A61B 5/0042; G01R 33/5608; A61B 2576/026

(57) ABSTRACT

A system and method are provided for determining a distribution of cellular populations in a patient. The method includes accessing neurite orientation dispersion and density imaging (NODDI) data acquired from the patient and performing a topological data analysis of the NODDI data to generate topological information. The method also includes correlating the topological information with cell population information and generating a report including the distribution of cellular populations in the patient based on the correlating of the topological information with cell population information.

### CROSS REFERENCE TO RELATED APPLICATIONS

N/A

### STATEMENT REGARDING FEDERALLY SPONSORED RESEARCH

N/A

### BACKGROUND

The field of the invention is systems and methods for magnetic resonance imaging (“MRI”). More particularly, the invention relates to systems and methods for using MRI to deconvolve cellular population, such as in the brain.

Medical imaging is a powerful and integral part of modern clinical medicine. Magnetic resonance imaging (MRI), in particular, is regularly relied upon to create anatomical images of patients with great resolution. In addition, MRI has the ability to provide some physiological information, such as when producing functional MRI (fMRI) images. This combination of anatomical images with substantial resolution and physiological information is regularly sought in a variety of clinical settings. The demand for information marrying great anatomical imaging with sensitive physiological imaging has led some manufactures to combine multiple imaging modalities, such as combining MRI with positron emission tomography (PET). PET systems are capable of acquiring physiological information that goes well beyond any information that can be acquired by MRI, or even other modalities, like computed tomography (CT) or ultrasound. Thus, these combination systems, like MR-PET systems, provide the advantage of acquiring the anatomical data using MRI and the physiological data using PET in a way that is perfectly registered in time and space. Unfortunately, the engineering limitations of combing two distinct modalities leads to tradeoffs in the overall functionality of the combination systems, when compared to individual MRI or PET systems. Furthermore, the physiological information that can be acquired is limited to that of the combined imaging modality and, in the case of PET, radiotracer (e.g., metabolic uptake). As such, even when such specialized system are available, a multitude of additional physiological testing is generally performed and must be somehow synthesized with the imaging information.

In addition to whatever anatomical and physiological information is acquired, modern clinical medicine also, generally, requires patient historical information and, increasingly, patient genetic information. As such, whether or not the anatomical and physiological information is combined by the imaging hardware, as is the case in a combined MR-PET system, or done manually by the clinician, the clinician must synthesize the anatomical and physiological information with the patient history and genetic information. Therefore, clinical decision making is often a function of synthesizing a variety of disparate information.

Thus, it would be desirable to have systems and methods that empower clinicians to make better healthcare decisions by improving the information available without just adding one more disparate report to an already extensive pile.

### SUMMARY

The present disclosure overcomes the aforementioned drawbacks by providing systems and methods for deconvolving cellular population in a patient. More particular, the systems and methods provided herein use quantitative neuroimaging data fed into a trained computational model to determine the percentage of various cell types. For a given region of interest, the systems and methods provided herein can determine the percentage of various cell types, such as neurons and glia (e.g., microglia, astrocytes, oligodendrocytes).

In accordance with one aspect of the disclosure, a method is provided for determining a distribution of cellular populations in a patient. The method includes accessing neurite orientation dispersion and density imaging (NODDI) data acquired from the patient and performing a topological data analysis of the NODDI data to generate topological information. The method also includes correlating the topological information with cell population information and generating a report including the distribution of cellular populations in the patient based on the correlating of the topological information with cell population information.

In accordance with another aspect of the disclosure, a magnetic resonance imaging (MRI) system is provided that includes a magnet system configured to generate a polarizing magnetic field about at least a portion of a subject arranged in the MRI system, a plurality of gradient coils configured to apply a gradient field to the polarizing magnetic field, and a radio frequency (RF) system configured to apply an excitation field to the subject and acquire MR image data from the subject. The system also includes a computer system programmed to control the plurality of gradient coils and the RF system to acquire neurite orientation dispersion and density imaging (NODDI) data from the subject, access the NODDI data acquired from the subject, perform a topological data analysis of the NODDI data to generate topological information, correlate the topological information with cell population information, and generate a report including the distribution of cellular populations in the patient based on the correlating of the topological information with cell population information.

In accordance with yet another aspect of the disclosure a computer readable storage medium is provided having instructions stored thereon that, when executed by a computer processor, cause the computer processor to carry out a process for determining a distribution of cellular populations in a patient. The process includes accessing neurite orientation dispersion and density imaging (NODDI) data acquired from the patient, performing a topological data analysis of the NODDI data to generate topographical information, correlating the topological information with cell population information, and generating a report including the distribution of cellular populations in the patient based on the correlating of the topological information with cell population information.

The foregoing and other aspects and advantages of the invention will appear from the following description. In the description, reference is made to the accompanying drawings, which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram of an exemplary magnetic resonance imaging (“MRI”) system configured in accordance with the present disclosure.

FIG. 2 is a graphic illustration of a tissue construct in accordance with the present disclosure.

FIG. 3 a graphic representation of a process for generating persistence statistics.

FIG. 4 is a schematic diagram of a platform in accordance with the present disclosure.

FIG. 5 is a flow chart setting forth steps of a process in accordance with the present disclosure.

FIG. 6 is a schematic diagram of a system in accordance with the present disclosure.

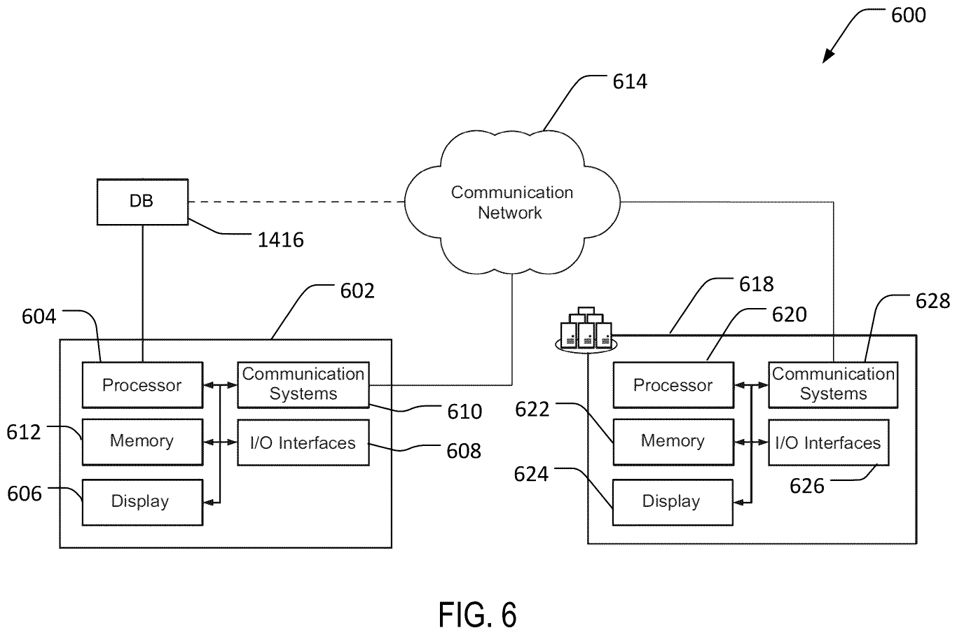

### DETAILED DESCRIPTION

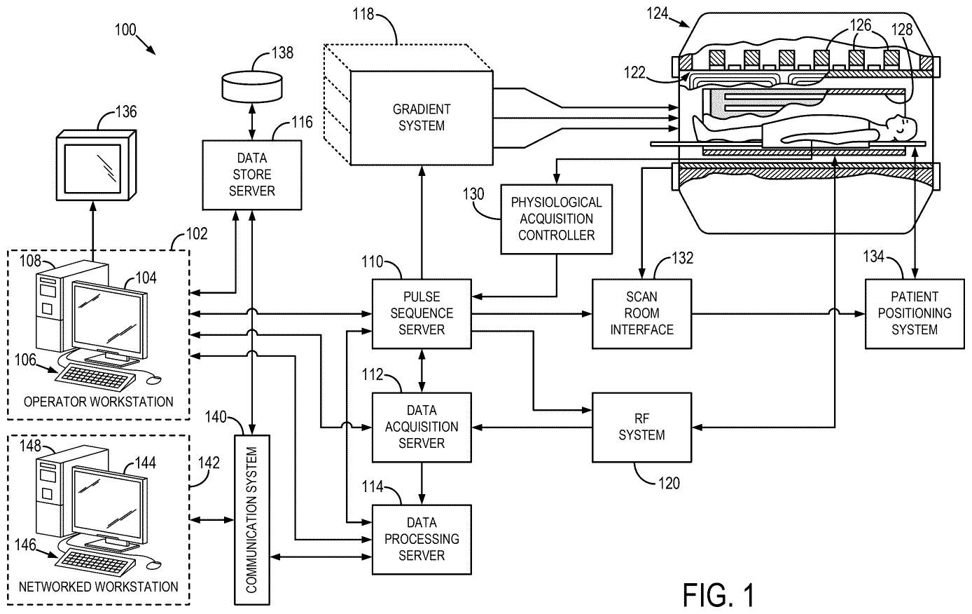

The systems and methods provided herein may be performed using MRI system, MRI data, or other computerized systems. As but one example, referring now to FIG. 1, a magnetic resonance imaging (“MRI”) system 100 is provided that configured to carry out the processes and techniques described herein. The MRI system 100 includes an operator workstation 102, which will typically include a display 104, one or more input devices 106 (such as a keyboard and mouse or the like), and a processor 108. The processor 108 may include a commercially available programmable machine running a commercially available operating system. The operator workstation 102 provides the operator interface that enables scan prescriptions to be entered into the MRI system 100. In general, the operator workstation 102 may be coupled to multiple servers, including a pulse sequence server 110; a data acquisition server 112; a data processing server 114; and a data store server 116. The operator workstation 102 and each server 110, 112, 114, and 116 are connected to communicate with each other. For example, the servers 110, 112, 114, and 116 may be connected via a communication system 140, which may include any suitable network connection, whether wired, wireless, or a combination of both. As an example, the communication system 140 may include both proprietary or dedicated networks, as well as open networks, such as the internet.

The pulse sequence server 110 functions in response to instructions downloaded from the operator workstation 102 to operate a gradient system 118 and a radiofrequency (“RF”) system 120. Gradient waveforms to perform the prescribed scan are produced and applied to the gradient system 118, which excites gradient coils in an assembly 122 to produce the magnetic field gradients Gx, Gy, Gz used for position encoding magnetic resonance signals. The gradient coil assembly 122 forms part of a magnet assembly 124 that includes a polarizing magnet 126 and a whole-body RF coil 128.

RF waveforms are applied by the RF system 120 to the RF coil 128, or a separate local coil (not shown in FIG. 1), in order to perform the prescribed magnetic resonance pulse sequence. Responsive magnetic resonance signals detected by the RF coil 128, or a separate local coil, are received by the RF system 120, where they are amplified, demodulated, filtered, and digitized under direction of commands produced by the pulse sequence server 110. The RF system 120 includes an RF transmitter for producing a wide variety of RF pulses used in MRI pulse sequences. The RF transmitter is responsive to the scan prescription and direction from the pulse sequence server 110 to produce RF pulses of the desired frequency, phase, and pulse amplitude waveform. The generated RF pulses may be applied to the whole-body RF coil 128 or to one or more local coils or coil arrays.

The RF system 120 also includes one or more RF receiver channels. Each RF receiver channel includes an RF preamplifier that amplifies the magnetic resonance signal received by the coil 128 to which it is connected, and a detector that detects and digitizes the I and Q quadrature components of the received magnetic resonance signal. The magnitude of the received magnetic resonance signal may, therefore, be determined at any sampled point by the square root of the sum of the squares of the I and Q components:

$$M = \sqrt{{I}^{2} + {Q}^{2}} ; \qquad Eqn . 1$$

and the phase of the received magnetic resonance signal may also be determined according to the following relationship:

$$φ = {tan}^{- 1} ( \frac{Q}{I} ) . \qquad Eqn . 2$$

The pulse sequence server 110 also optionally receives patient data from a physiological acquisition controller 130. By way of example, the physiological acquisition controller 130 may receive signals from a number of different sensors connected to the patient, such as electrocardiogramaignals from electrodes, or respiratory signals from a respiratory bellows or other respiratory monitoring device. Such signals are typically used by the pulse sequence server 110 to synchronize, or “gate,” the performance of the scan with the subject's heartbeat or respiration.

The pulse sequence server 110 also connects to a scan room interface circuit 132 that receives signals from various sensors associated with the condition of the patient and the magnet system. It is also through the scan room interface circuit 132 that a patient positioning system 134 receives commands to move the patient to desired positions during the scan.

The digitized magnetic resonance signal samples produced by the RF system 120 are received by the data acquisition server 112. The data acquisition server 112 operates in response to instructions downloaded from the operator workstation 102 to receive the real-time magnetic resonance data and provide buffer storage, such that no data is lost by data overrun. In some scans, the data acquisition server 112 does little more than pass the acquired magnetic resonance data to the data processor server 114. However, in scans that require information derived from acquired magnetic resonance data to control the further performance of the scan, the data acquisition server 112 is programmed to produce such information and convey it to the pulse sequence server 110. For example, during prescans, magnetic resonance data is acquired and used to calibrate the pulse sequence performed by the pulse sequence server 110. As another example, navigator signals may be acquired and used to adjust the operating parameters of the RF system 120 or the gradient system 118, or to control the view order in which k-space is sampled. In still another example, the data acquisition server 112 may also be employed to process magnetic resonance signals used to detect the arrival of a contrast agent in a magnetic resonance angiography (“MRA”) scan. By way of example, the data acquisition server 112 acquires magnetic resonance data and processes it in real-time to produce information that is used to control the scan.

The data processing server 114 receives magnetic resonance data from the data acquisition server 112 and processes it in accordance with instructions downloaded from the operator workstation 102. Such processing may, for example, include one or more of the following: reconstructing two-dimensional or three-dimensional images by performing a Fourier transformation of raw k-space data; performing other image reconstruction techniques, such as iterative or backprojection reconstruction techniques; applying filters to raw k-space data or to reconstructed images; generating functional magnetic resonance images; calculating motion or flow images; and so on.

Images reconstructed by the data processing server 114 are conveyed back to the operator workstation 102. Images may be output to operator display 112 or a display 136 that is located near the magnet assembly 124 for use by attending clinician. Batch mode images or selected real time images are stored in a host database on disc storage 138. When such images have been reconstructed and transferred to storage, the data processing server 114 notifies the data store server 116 on the operator workstation 102. The operator workstation 102 may be used by an operator to archive the images, produce films, or send the images via a network to other facilities.

The MRI system 100 may also include one or more networked workstations 142. By way of example, a networked workstation 142 may include a display 144, one or more input devices 146 (such as a keyboard and mouse or the like), and a processor 148. The networked workstation 142 may be located within the same facility as the operator workstation 102, or in a different facility, such as a different healthcare institution or clinic. The networked workstation 142 may include a mobile device, including phones or tablets.

The networked workstation 142, whether within the same facility or in a different facility as the operator workstation 102, may gain remote access to the data processing server 114 or data store server 116 via the communication system 140. Accordingly, multiple networked workstations 142 may have access to the data processing server 114 and the data store server 116. In this manner, magnetic resonance data, reconstructed images, or other data may exchanged between the data processing server 114 or the data store server 116 and the networked workstations 142, such that the data or images may be remotely processed by a networked workstation 142. This data may be exchanged in any suitable format, such as in accordance with the transmission control protocol (“TCP”), the internet protocol (“IP”), or other known or suitable protocols.

As described above, current methodologies for assessment of non-anatomical information using imaging systems like MRI are encumbered by significant limitations, including low specificity, inability to accurately quantify, and low biocompatibility/toxicity. For example, some have attempted to discern inflammation in the brain using diffusion tensor imaging (DTI) MRI. DTI MRI is successfully used with regularity to assess structures in the brain, such as white matter fibers. However, when attempting to assess neuro-inflammation using DTI, the lack of specificity inhibits clinical utility. Others have combined PET or other tracer-based imaging techniques with MRI. However, such efforts often struggle to provide quantitative information needed by clinicians. Further still, some have attempted to utilize microparticles of iron oxide (MPIO) to target particular physiological processes with enhanced contrast using MRI. Unfortunately, such MPIO agents carry biocompatibility and/or toxicity concerns that limit utility. Even beyond all these limitations, none of these efforts provide quantitative information about the cell types in the area of interest. However, as will be described herein, the present disclosure provides systems and methods to deconvolve cellular population. For a given region of interest, the systems and methods provided herein can determine the percentage of various cell types, such as neurons and glia (e.g., microglia, astrocytes, oligodendrocytes.) More particular, the systems and methods provided herein use quantitative neuroimaging data fed into a trained computational model to determine the percentage of various cell types. On one non-limiting example, systems and methods are provided to perform a topological data analysis of MR neurite orientation dispersion and density imaging (NODDI) data and then process the data to generate a report with deconvolved cellular population information.

Diffusion MRI can be used to measure tissue microstructure directly. One such approach is a model-based strategy in which a geometric model of the microstructure of interest predicts the MR signal from water diffusion within the tissue. A multi-compartment tensor models stands in contrast with DTI, which employs a single-compartment diffusion tensor model. As described herein, the multi-compartment model can be used to quantitatively express how the total normalized diffusion MRI signal is comprised by: (1) anisotropic diffusion within neuronal process and (2) anisotropic diffusion arising from around these processes. Some attempts to make multi-compartment diffusion models focused on the formulation and subsequent validation of mathematical models of water diffusion in neurites to garner estimates of neurite orientation as well as neurite density. Subsequent quantitative comparisons following co-registration of MR data with histology and light and electron microscopy demonstrated the relationship between the intracellular (intra-neurite) MR diffusion tensor and axonal/dendritic architecture.

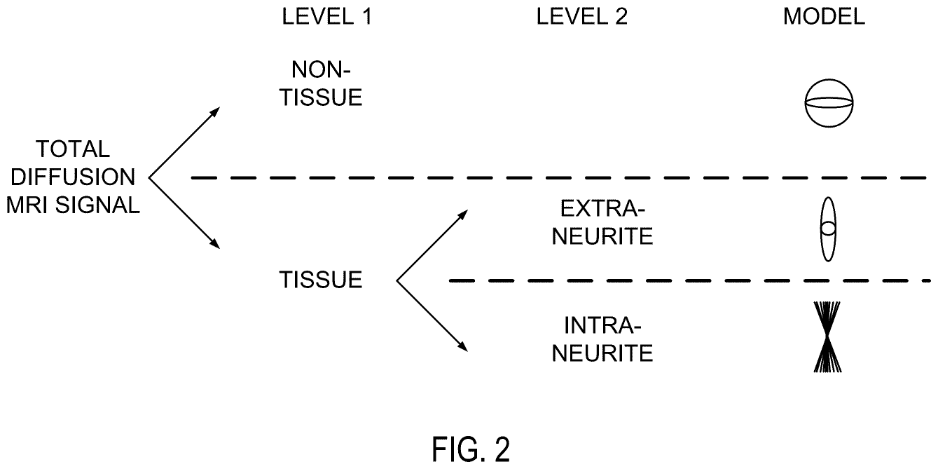

As illustrated in FIG. 2, the present disclosure provides a neurite orientation dispersion and density imaging (NODDI) model that advances multi-compartment diffusion imaging as a clinically feasible imaging technique. To generate greater tissue specificity than standard DWI techniques such as DTI, NODDI employs a model-based strategy designed to measure water diffusion arising from distinct tissue compartments. Specifically, FIG. 1A provides a NODDI tissue model in accordance with the present disclosure. The NODDI tissue model is a multi-compartmental model of the total normalized diffusion MRI signal and comprises: (1) non-tissue (Fᵢₛₒ); (2) extraneurite (orientation dispersion index, ODI); and (3) intraneurite (neurite density index, NDI). Non-tissue material, such as cerebral spinal fluid (CSF), represents a first level (level 1) of the model and can be modeled as a volume. Also at level 1 is tissue. However, unlike traditional models that models tissue as a single signal, the present disclosure includes a second level (level 2) that divides signal that otherwise would be attributed to “tissue” to be formed as extra-neurite material, such as cell bodies and glial cells (ODI) and intra-neurite material, such as axons and dendrites (NDI).

In the NODDI model, diffusivity in the extra-neurite compartment is measured by ODI, which was originally conceptualized to measure how changes in neurite dispersion influence water diffusivity in the extra-neurite space without accounting for the potential contribution that glial cells (such as microglia) can have on quantitative measures of ODI. However, within the extra-neurite compartment, glial cells reside, which account for a large percentage of non-neuronal cells. As microglia have been found to comprise 5-15% of all glial cells and, in response to inflammatory stimuli, undergo substantial changes in both morphology and density, these changes would be expected to significantly alter the degree of hindered diffusion in the extra-neurite compartment. These changes offer a potential opportunity to assess microglial activation and microglialmediated neuroinflammation by probing water diffusion using a modality such as MRI, but only if a model is utilized that enables the proper consideration of the underlying mechanisms.

The present disclosure recognizes that the NODDI model of FIG. 2 distinguishes three microstructural environments, including the intracellular, extracellular, and CSF compartments. The intracellular compartment (NDI) is defined by the space bounded by the membrane of neurites. The extracellular compartment (ODI) is defined by the space around the neurites, which includes neural cell bodies (somas) as well as glial cells.

Multi-compartment diffusion models biophysically model the total DWI signal as a sum of the diffusion weighted signal arising from a combination of biophysical compartments with different underlying cellular microstructures:

$$S = {S}_{0} {\sum }_{i = 0}^{n} {w}_{i} {S}_{i} ; \qquad Eqn . 3$$

where S₀ is the signal for the non-diffusion weighted (or b0) acquisitions, wᵢ is the volume fraction, and Si is the signal function for the ith of n total compartments. In the NODDI model in accordance with the present disclosure, the diffusion MRI signal is described as a sum of three non-exchanging biophysical compartments:

$$S = ( 1 - {v}_{iso} ) ( {v}_{ic} {S}_{ic} + ( 1 - {v}_{ic} ) {S}_{ec} ) + {v}_{iso} {S}_{iso} ; \qquad Eqn . 4$$

where S is the entire normalized signal; S_{ic}, S_{ec}, and Sᵢₛₒ are the normalized signals of the intracellular, extracellular, and CSF compartments, respectively, and Vic and vᵢₛₒ are the normalized volume fractions of the intracellular and CSF compartments.

With this multi-compartment model and the underlying anatomical information inherent to MRI data, the present disclosure recognizes that, if the NODDI images could be parameterized, the images could then be processed to elicit information about the underlying cellular populations.

Newer methods, such as radiomics, have tried to provide a way to parameterize images using texture analysis. However, these methods have not been well-suited to algorithmic or machine-learning processing due to the multicollinearity of features produced. Essentially, most of these features can be collapsed into 2 or 3 principle components, which does not provide enough variability to predict cell population percentages. This multicollinearity is due to the fact many radiomic features use the same underlying mathematical formulas, only modifying certain parameters.

Topology is a branch of mathematics focused on the properties of shapes that can be stretched, bent, twisted, or shrunk without being broken or affixed together. “Topology,” as applied to imaging, is essentially the study of shapes through its overall connectedness (number of loops and holes), rather than through geometric measurements (number of angles, number of vertices, distances between vertices, etc.). One can warp an object geometrically (i.e., shrink, expand, twist it) without changing “topology”, but opening new holes or filling in holes is not permissible under the topology rubric. Thus, geometry focuses on precise measurements like distance and angles, while topology examines properties that remain unchanged under continuous deformations like stretching, bending, or twisting. The present disclosure recognizes that topological processing provides a basis to mathematically distinguish shapes from each other that could not be distinguished using classical geometry.

Within topology, Betti numbers are used to distinguish topological spaces based on the connectivity of n-dimensional simplicial complexes. The present disclosure recognizes that Betti numbers of a given dataset can be tracked to discern a “persistence.”

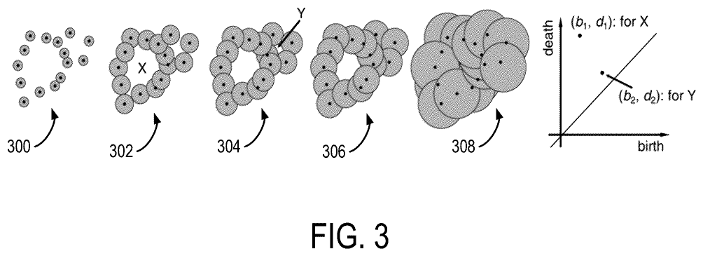

For example, a process can be performed that starts by creating a radius around each point in the dataset. As the radius increases, the process tracks when rings or spheres in the data first appear (i.e., are “born”). Referring to FIG. 3, at instance 10, no ring is present. However, at instance 12 a ring, denoted “X” appears with r=b₁. Then, at instance 14, a second ring, denoted “Y” appears with r=b₂. Once a hole is covered by the surrounding radii, the feature “dies.” For example, at instance 16, ring Y disappears with r=d₂. Then, at instance 18, ring X disappears with r=d₁. The persistence of a feature is therefore defined as its lifetime (death time-birth time). Once all features are killed the process stops A plot of birth vs. death of each feature provides the “persistence diagram” that describes the topology of a multidimensional dataset.

With this in mind, the process can compare the topology of different datasets by computing statistics from the birth, death, and persistence distributions of the different classes of Betti numbers. Importantly, these features are less multicollinear as they describe the topology across different dimensions. Also, it is noted that, as a point itself is considered a connected component (i.e., a B0 feature), all B0 features are born at 0 but die at unique times (when they connect with other points).

By using topological data analysis (TDA), the systems and methods provided herein can detect the existence and extent of noise while still maintaining the overall distribution of topological features. This is because the topological features of objects that are warped are still the same. This allows the creation of processes that are robust to noise inherent to the collection of MRI images as well as to transformations when warping images to the same template, while still being able to detect small differences between images.

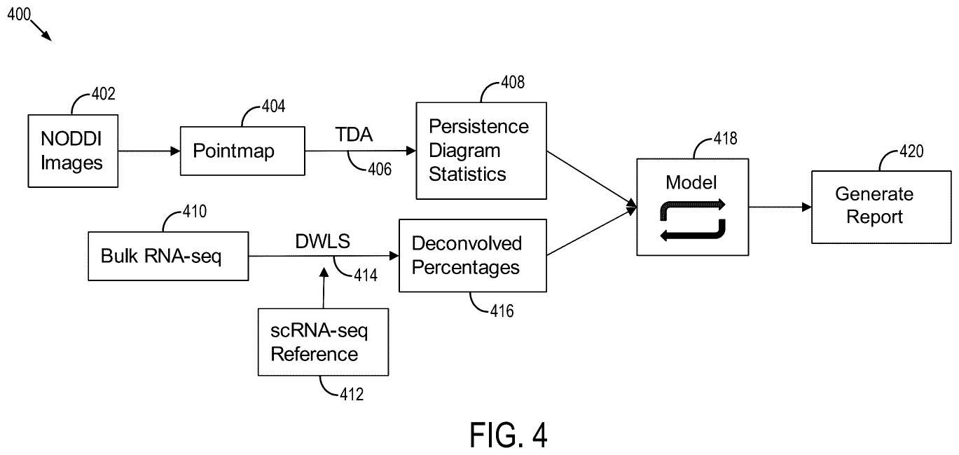

Leveraging these constructs, the present disclosure provides systems and methods for generating a model for deconvolving cellular populations. Referring to FIG. 4, a pipeline 400 is provided that begins with the access of NODDI images from computer storage or acquisition of NODDI images using a system such as described above with respect to FIG. 1. That is, at process block 402, previously acquired NODDI images are accessed or NODDI data is acquired using an MRI system and reconstructed into images. Then, at block 404, pointmaps are created, such as described above with respect to FIG. 2. Topological data analysis (TDA) is performed 406 to generate persistence diagram statistics at block 408, such as described above with respect to FIG. 3. Persistence diagram statistics from a NODDI training set can be scaled and matched to corresponding cell population percentages from age and sex-matched RNA-seq samples, as will be described. Statistics from both NDI and ODI images can be used.

In parallel, at block 410, bulk RNA-seq data can be accessed or acquired. The bulk RNA-seq samples may be from neuronal tissue, or a different tissue of interest. A single cell RNA-seq reference dataset (scRNA-seq reference data) can also be acquired or accessed at block 412. At 414, dampened weighted least squares (DWLS) estimation is performed for gene expression deconvolution. DWLS can computationally infer the cell-type composition of a bulk RNA-seq data set. In this way, a reference set of data is provided that includes deconvolved cell population percentages at process block 416.

With the persistence diagram statistics and the deconvolved percentages in hand, an initial training of a model 418 can be performed. The model 418, as described herein may be referred to as a the XGBoost model. In one non-limiting example, permutation sampling and leave-one-out cross validation can be used to train the model to associate cell population percentages with combinations of TDA features.

Once trained, the model 418 can generate a report at block 420. Alternatively, instead of training the model 418, the model 418 may be algorithmically built from analytical processing and correlation of the persistence diagram (or other output of TDA processing) and deconvolved cellular population information. In either case, the report include a predicted percentage from the persistence diagram statistics of novel NODDI images, without having to sequence any new RNA-seq samples.

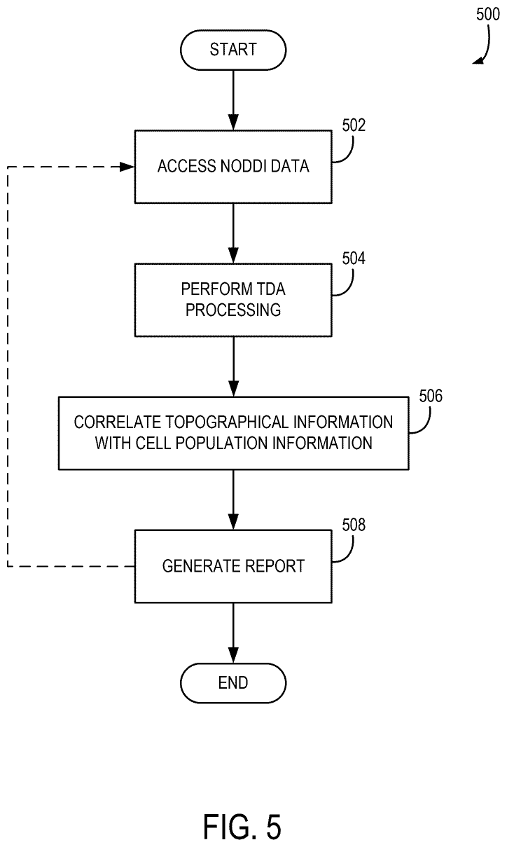

Referring now to FIG. 5, a process 500 for clinical use of the systems and methods provided herein is provided. At process block 502, NODDI data is accessed. As described above, such data may be acquired using an MRI system or may be accessed from storage. Then, at process block 504, TDA processing is performed. For example, pointmaps may be generated from NODDI images to then generate topological information, such as persistence diagrams as described above. Then, at process block 506, the topological information is then correlated with cell population information. Correlation may include using a trained model, such as the XGBoost model described above. Alternatively, correlation may be performed using a traditional algorithm, for example that compares against a database of cell population percentages correlated to, for example, persistence diagram statistics and/or other differentiated statistics derived from NODDI images. Regardless of the particular correlation or comparison mechanism utilized, at process block 508, a report is generated, which can include a report of the deconvolved cellular population information of the cells reflected in the NODDI data from process block 502.

The above process can be repeated periodically to empower noninvasive identification and tracking of cell populations in the brain over time, or over a treatment course. This may be useful in tracking therapies, evaluating efficacy of therapies, or collecting data or drug discovery or testing. For example, having cell-specific insights enables assessment of disease processes or drug therapies. As a further example, in treating a patient with multiple sclerosis, an inflammatory disease that attacks oligodendrocytes, this method will be able to detect if a therapy is working by directly interrogating if oligodendrocyte populations are being replenished and also by detecting if neuroinflammation is going down with evidence of decrease microglia cell populations.

Data from any clinical MR scanner can be supplied to a software program that computes the cell population quantities. Thus, the systems and methods described above, can be implemented in a variety of configurations. In one non-limiting example, a system 600 can be used to process and analyze as disclosed herein. For example, the system 600 can include a computing device 602. The computing device 602 can be a phone, workstation (such as described above, a head mounted display, a personal computer, a calculator, smart glasses, a gaming console, a table, or the like. The computing device 602 can include a processor 604, a display 606, one or more inputs/output interfaces 608, one or more communication systems 610, and/or memory 612. In some embodiments, processor 1404 can be any suitable hardware processor or combination of processors, such as a central processing unit (CPU), a graphics processing unit (GPU), an application specific integrated circuit (ASIC), a field-programmable gate array (FPGA), etc. In some embodiments, the display 1406 can include any suitable display device, such as a computer monitor, a touchscreen, a television, a head-mounted display, a tablet, etc. In some embodiments, the inputs 608 can include any suitable input devices and/or sensors that can be used to receive user input, such as a keyboard, a mouse, a touchscreen, a microphone, a camera, or the like.

In some examples, the communication systems 610 can include any of a variety of suitable hardware, firmware, or software for communicating information over the communication network 614 and/or other suitable communication networks. For example, the communication systems 610 can include one or more transceivers, one or more communication chips and/or chip sets, etc. In a more particular example, the communication systems 610 can include hardware, firmware and/or software that can be used to establish a Wi-Fi connection, a Bluetooth connection, a cellular connection, an Ethernet connection, or the like.

In some configurations, the memory 612 can include any suitable storage device or devices that can be used to store instructions, values, data, etc., that can be used, for example, to perform image processing. The memory 612 can include any of a variety of suitable volatile memory, non-volatile memory, storage, or any suitable combination thereof. For example, the memory 612 can include random access memory (RAM), read-only memory (ROM), electronically-erasable programmable read-only memory (EEPROM), one or more flash drives, one or more hard disks, one or more solid state drives, one or more optical drives, or the like. In some configurations, the memory 612 can have encoded thereon a computer program for controlling operation of the computing device 602.

As shown, the system 600 can include a database 616. The database 616 can include stored data, including NODDI images or data, sequence data, a trained model, an algorithm or the like. The system 600 can further include remote computing devices 618. Remote computing devices 618 can include cloud infrastructure, for example, and can have the capacity for computationally intensive operations that cannot practically be done on a user's personal device. The remote computing device 618 can be a single device, a server, a virtual server, a distributed computing system, or any know arrangement of computing hardware and software. A shown, the remote computing device 618 can include a processor 620, memory 622, display 624, Input/output interfaces 626, communications systems 628 and the like.

One or more embodiments are described and illustrated in the following description and accompanying drawings. These embodiments are not limited to the specific details provided herein and may be modified in various ways. Furthermore, other embodiments may exist that are not described herein. Also, the functionality described herein as being performed by one component may be performed by multiple components in a distributed manner. Likewise, functionality performed by multiple components may be consolidated and performed by a single component. Similarly, a component described as performing particular functionality may also perform additional functionality not described herein. For example, a device or structure that is “configured” in a certain way is configured in at least that way, but may also be configured in ways that are not listed. Furthermore, some embodiments described herein may include one or more electronic processors configured to perform the described functionality by executing instructions stored in non-transitory, computer-readable medium. Similarly, embodiments described herein may be implemented as non-transitory, computer-readable medium storing instructions executable by one or more electronic processors to perform the described functionality. As used in the present application, “non-transitory, computer readable medium” comprises all computer-readable media but does not consist of a transitory, propagating signal. Accordingly, non-transitory computer-readable medium may include, for example, a hard disk, a CD-ROM, an optical storage device, a magnetic storage device, a ROM (Read Only Memory), a RAM (Random Access Memory), register memory, a processor cache, or any combination thereof.

In addition, the phraseology and terminology used herein is for the purpose of description and should not be regarded as limiting. For example, the use of “including,” “containing,” “comprising,” “having,” and variations thereof herein is meant to encompass the items listed thereafter and equivalents thereof as well as additional items. The terms “connected” and “coupled” are used broadly and encompass both direct and indirect connecting and coupling. Further, “connected” and “coupled” are not restricted to physical or mechanical connections or couplings and can include electrical connections or couplings, whether direct or indirect. In addition, electronic communications and notifications may be performed using wired connections, wireless connections, or a combination thereof and may be transmitted directly or through one or more intermediary devices over various types of networks, communication channels, and connections. Moreover, relational terms such as first and second, top and bottom, and the like may be used herein solely to distinguish one entity or action from another entity or action without necessarily requiring or implying any actual such relationship or order between such entities or actions.

The phrase “such as” should be interpreted as “for example, including.” Moreover, the use of any and all exemplary language, including but not limited to “such as”, is intended merely to better illuminate the disclosed technology and does not pose a limitation on the scope of the disclosed technology unless otherwise claimed. As used in this specification and the claims, the singular forms “a,” “an,” and “the” include plural forms unless the context clearly dictates otherwise.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The above-described system may be configured or otherwise used to carry out processes in accordance with the present disclosure. In particular, as will be described in further detail, The present invention has been described in terms of one or more preferred embodiments, and it should be appreciated that many equivalents, alternatives, variations, and modifications, aside from those expressly stated, are possible and within the scope of the invention.

1. A method for determining a distribution of cellular populations in a patient, the method comprising:
accessing neurite orientation dispersion and density imaging (NODDI) data acquired from the patient;
performing a topological data analysis of the NODDI data to generate topological information;
correlating the topological information with cell population information; and
generating a report including the distribution of cellular populations in the patient based on the correlating of the topological information with cell population information.

2. The method of claim 1, wherein performing the topological data analysis includes generating a pointmap from the NODDI data.

3. The method of claim 1, wherein performing the topological data analysis includes developing persistence diagram statistics using the NODDI data.

4. The method of claim 1, wherein correlating the topological information with cell population information includes delivering the topological information to a model trained using RNA-seq data.

5. The method of claim 1, wherein correlating the topological information with cell population information includes comparing the topological information to a database of deconvolved cellular percentages.

6. The method of claim 1, wherein the NODDI data acquired from the patient includes NODDI data from a brain of the patient.

7. A magnetic resonance imaging (MRI) system comprising:
a magnet system configured to generate a polarizing magnetic field about at least a portion of a subject arranged in the MRI system;
a plurality of gradient coils configured to apply a gradient field to the polarizing magnetic field;
a radio frequency (RF) system configured to apply an excitation field to the subject and acquire MR image data from the subject;
a computer system programmed to:
control the plurality of gradient coils and the RF system to acquire neurite orientation dispersion and density imaging (NODDI) data from the subject;
access the NODDI data acquired from the subject;
perform a topological data analysis of the NODDI data to generate topological information;
correlate the topological information with cell population information; and
generate a report including the distribution of cellular populations in the patient based on the correlating of the topological information with cell population information.

8. The system of claim 7, wherein the computer system is further programmed to perform the topological data analysis by generating a pointmap from the NODDI data.

9. The system of claim 7, wherein the computer system is further programmed to perform the topological data analysis by developing persistence diagram statistics using the NODDI data.

10. The system of claim 7, wherein the computer system is further programmed to correlate the topological information with cell population information by delivering the topological information to a model trained using RNA-seq data.

11. The system of claim 7, wherein the computer system is further configured to correlate the topological information with cell population information by comparing the topological information to a database of deconvolved cellular percentages.

12. The system of claim 7, wherein the NODDI data acquired from the patient includes NODDI data from a brain of the patient.

12. A computer readable storage medium having instructions stored thereon that, when executed by a computer processor, cause the computer processor to carry out a process for determining a distribution of cellular populations in a patient comprising:
accessing neurite orientation dispersion and density imaging (NODDI) data acquired from the patient;
performing a topological data analysis of the NODDI data to generate topographical information;
correlating the topological information with cell population information; and
generating a report including the distribution of cellular populations in the patient based on the correlating of the topological information with cell population information.

13. The computer readable storage medium of claim 12, wherein, to perform the topological data analysis, the computer process is further caused to generate a pointmap from the NODDI data.

14. The computer readable storage medium of claim 12, wherein, to perform the topological data analysis, the computer process is further caused to develop persistence diagram statistics using the NODDI data.

15. The computer readable storage medium of claim 12, wherein, to correlate the topological information with cell population information, the computer process is further caused to deliver the topological information to a model trained using RNA-seq data.

16. The computer readable storage medium of claim 12, wherein, to correlate the topological information with cell population information, the computer process is further caused to compare the topological information to a database of deconvolved cellular percentages.

17. The computer readable storage medium of claim 12, wherein the NODDI data acquired from the patient includes NODDI data from a brain of the patient.
